(19) United States
(12) Patent Application Publication
(10) Pub. No.: US 2010/0134759 A1
(43) Pub. Date: Jun. 3, 2010

(54) DIGITAL IMAGING SYSTEM FOR EYE PROCEDURES

(76) Inventors: Thomas A. Silvestrini, Alamo, CA (US); Steven John, Fremont, CA (US); Jose Garcia, Fremont, CA (US); Richard S. Lilly, San Jose, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/492,085

(22) Filed: Jun. 25, 2009

Related U.S. Application Data
(60) Provisional application No. 61/076,114, filed on Jun. 26, 2008.

(51) Int. Cl. A61B 3/117; A61B 3/14

(52) U.S. Cl. 351/206; 351/219

(57) ABSTRACT

Described herein is a hand-held gonioscopic imaging system that can be used to continuously display, capture and record images of the iridocorneal angle within the eye during implantation procedures. The system can be used, for example, during device implantation procedures for the treatment of glaucoma such that landmark identification continues during implantation. Intuitive real-time images viewed through the imaging systems described herein appear to the user to move in the same horizontal orientation as the instrument is actually being moved. The systems described herein also provide independent illumination sources for the camera and the surgical microscope that also have independent illumination controls.

### REFERENCE TO PRIORITY DOCUMENT

This application claims priority of U.S. Provisional Patent Application Ser. No. 61/076,114, entitled “Digital Imaging System for Eye Procedures” by Thomas Silvestrini, filed Jun. 26, 2008. Priority of the filing date of Jun. 26, 2008 is hereby claimed, and the disclosure of the Provisional Patent Application is hereby incorporated by reference.

### BACKGROUND

This disclosure relates generally to methods and devices for the treatment of glaucoma. In particular, disclosed is a hand-held digital imaging system that can be used to conveniently view the internal structures of the eye, such as the iridocorneal angle of the anterior chamber during surgical eye procedures such as implantation of a shunt to treat glaucoma.

The mechanisms that cause glaucoma are not completely known. It is known that glaucoma results in abnormally high pressure in the eye, which leads to optic nerve damage. Over time, the increased pressure can cause damage to the optic nerve, which can lead to blindness. Treatment strategies have focused on keeping the intraocular pressure down in order to preserve as much vision as possible over the remainder of the patient's life.

Past treatment includes the use of drugs that lower intraocular pressure through various mechanisms. The glaucoma drug market is an approximate two billion dollar market. The large market is mostly due to the fact that there are not any effective surgical alternatives that are long lasting and complication-free. Unfortunately, drug treatments need much improvement, as they can cause adverse side effects and often fail to adequately control intraocular pressure. Moreover, patients are often lackadaisical in following proper drug treatment regimens, resulting in a lack of compliance and further symptom progression.

With respect to surgical procedures, one way to treat glaucoma is to implant a drainage device in the eye. The drainage device functions to drain aqueous humor from the anterior chamber and thereby reduce the intraocular pressure. The drainage device is typically implanted using an invasive surgical procedure. Pursuant to one such procedure, a flap is surgically formed in the sclera. The flap is folded back to form a small cavity and the drainage device is inserted into the eye through the flap. Such a procedure can be quite traumatic as the implants are large and can result in various adverse events such as infections and scarring, leading to the need to re-operate.

Current devices and procedures for treating glaucoma have disadvantages and only moderate success rates. The procedures are very traumatic to the eye and also require highly accurate surgical skills, such as to properly place the drainage device in a proper location. In addition, the devices that drain fluid from the anterior chamber to a subconjunctival bleb beneath a scleral flap are prone to infection, and can occlude and cease working. This can require re-operation to remove the device and place another one, or can result in further surgeries. In view of the foregoing, there is a need for improved devices and methods for the treatment of glaucoma.

Gonioscopy refers to an examination of the angle structures in the anterior chamber of the eye. The angle of the eye is formed by the insertion of the peripheral iris into the wall of the eye. The angle includes a portion of the anterior ciliary body, the base of the iris processes, the trabeculum (uveoscleral, corneoscleral and juxtacanalicular meshworks), the scleral spur, Schlemm's canal, Schwalbe's line and the adjacent cornea.

A view of the angle can be important, for example, in diagnosing and monitoring eye conditions such as glaucoma as well as in the implantation of devices, for example drainage implants or shunts for the treatment of glaucoma. However, it is not possible to view the structures of the angle with direct observation. The scleral tissue projects anterior to the angle and the curvature of the cornea creates internal reflection when one attempts to view the angle obliquely. A device called a gonioscope or goniolens permits observation of the iridocorneal angle by placing a concave surface against the cornea eliminating the cornea as a refracting surface using obliquely inclined mirrors.

For procedures such as shunt implantation, identification of eye structures and landmarks are necessary for insuring proper placement of devices and preventing injury. A gonioscope and surgical microscope are typically used during implantation procedures. However, the gonioscope must be removed once the applier is inserted causing the physician to rely on memory of the region for the remainder of the procedure. Without visualizing through the gonioscope, the physician has no way of knowing the degree of accuracy in the implantation, for example, whether the dissection performed is above or below any particular anatomical landmark. Only after the implant is placed can the physician once again use a gonioscope to confirm accuracy and proper placement.

### SUMMARY

There is a need for an imaging system that is small enough to be hand-held that will also continuously display and capture images of structures within the eye during implantation of devices. There is also a need for an imaging system in which the images appear to the physician to move in the same horizontal orientation as the instrument is actually being moved.

In an embodiment, disclosed is a hand-held system for viewing the interior of a patient's eye. The system includes a viewing lens having a corneal contact surface, an anterior viewing surface and an optically transparent body therebetween. The optically transparent body includes a first internal planar surface and a second internal planar surface, the first and second internal planar surfaces each having a mirrored coating. The system also includes an illumination source and an imaging device.

Other features and advantages should be apparent from the following description of various embodiments, which illustrate, by way of example, the principles of the device and methods disclosed herein.

### BRIEF DESCRIPTION OF THE DRAWINGS

FIG. 1 is a cross-sectional, perspective view of a portion of the eye showing the anterior and posterior chambers of the eye.

FIG. 2 is a cross-sectional view of a human eye.

FIG. 3A is a perspective view of an exemplary viewing lens.

FIG. 3B shows an embodiment of a camera assembly.

FIG. 3C shows an embodiment of an imaging system including the assembly of FIG. 3B with the exemplary viewing lens of FIG. 3A attached thereto.

FIG. 4 shows an exemplary two mirrored viewing lens.

FIG. 5 is a schematic view of an embodiment of an imaging system.

FIG. 6 is a schematic view of another embodiment of an imaging system.

FIG. 7 shows an exemplary delivery system that can be used to deliver an implant into the eye using an embodiment of the imaging system.

FIG. 8 shows a cross-sectional view of the eye.

FIG. 9 shows the delivery system penetrating the eye while using a imaging system for visualization.

FIG. 10 shows an enlarged view of the anterior region of the eye with a portion of the delivery system positioned in the anterior chamber.

FIG. 11 shows the distal tip of the applier positioned within the suprachoroidal space.

### DETAILED DESCRIPTION

Described herein is a hand-held imaging system that can be used to continuously display, capture and record images of structures within a patient's eye, such as the iridocorneal angle, during implantation procedures. The system can be used, for example, during device implantation procedures for the treatment of glaucoma such that landmark identification continues during implantation. Intuitive real-time images viewed through the imaging systems described herein appear to the user to move in the same horizontal orientation as the instrument is actually being moved. The systems described herein also provide independent illumination sources for the camera and the surgical microscope that also have independent illumination controls.

### Exemplary Eye Anatomy

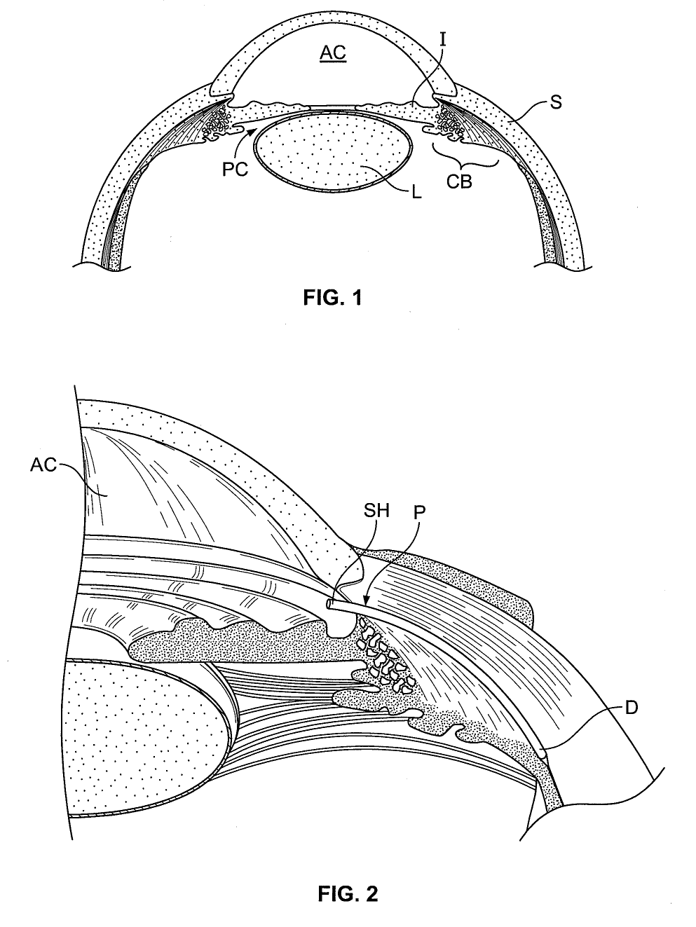

FIG. 1 is a cross-sectional view of a portion of the human eye. The eye is generally spherical and is covered on the outside by the sclera S. The retina lines the inside posterior half of the eye. The retina registers the light and sends signals to the brain via the optic nerve. The bulk of the eye is filled and supported by the vitreous body, a clear, jelly-like substance.

The elastic lens L is located near the front of the eye. The lens L provides adjustment of focus and is suspended within a capsular bag from the ciliary body CB, which contains the muscles that change the focal length of the lens. A volume in front of the lens L is divided into two by the iris I, which controls the aperture of the lens and the amount of light striking the retina. The pupil is a hole in the center of the iris I through which light passes. The volume between the iris I and the lens L is the posterior chamber PC. The volume between the iris I and the cornea is the anterior chamber AC. Both chambers are filled with a clear liquid known as aqueous humor.

The ciliary body CB continuously forms aqueous humor in the posterior chamber PC by secretion from the blood vessels. The aqueous humor flows around the lens L and iris I into the anterior chamber and exits the eye through the trabecular meshwork, a sieve-like structure situated at the corner of the iris I and the wall of the eye (the corner is known as the iridocorneal angle). Some of the aqueous humor filters through the trabecular meshwork near the iris root into Schlemm's canal, a small channel that drains into the ocular veins. A smaller portion rejoins the venous circulation after passing through the ciliary body and eventually through the sclera (the uveoscleral route).

Glaucoma is a disease wherein the aqueous humor builds up within the eye. In a healthy eye, the ciliary processes secrete aqueous humor, which then passes through the angle between the cornea and the iris. Glaucoma appears to be the result of clogging in the trabecular meshwork. The clogging can be caused by the exfoliation of cells or other debris. When the aqueous humor does not drain properly from the clogged meshwork, it builds up and causes increased pressure in the eye, particularly on the blood vessels that lead to the optic nerve. The high pressure on the blood vessels can result in death of retinal ganglion cells and eventual blindness.

Closed angle (acute) glaucoma can occur in people who were born with a narrow angle between the iris and the cornea (the anterior chamber angle). This is more common in people who are farsighted (they see objects in the distance better than those which are close up). The iris can slip forward and suddenly close off the exit of aqueous humor, and a sudden increase in pressure within the eye follows.

Open angle (chronic) glaucoma is by far the most common type of glaucoma. In open angle glaucoma, the iris does not block the drainage angle as it does in acute glaucoma. Instead, the fluid outlet channels within the wall of the eye gradually narrow with time. The disease usually affects both eyes, and over a period of years the consistently elevated pressure slowly damages the optic nerve.

Implantation of drainage devices, or shunts, in the eye can be used to treat glaucoma. The shunt provides a fluid pathway for flow or drainage of aqueous humor from the anterior chamber and thereby reduces the intraocular pressure. FIG. 2 is a cross-sectional, perspective view of a portion of the eye showing the anterior and posterior chambers of the eye including a shunt SH positioned inside the eye. A proximal end P of the shunt is located in or near the anterior chamber AC and a distal end D communicates with and/or is located in the suprachoroidal space (sometimes referred to as the perichoroidal. space). The suprachoroidal space can include the region between the sclera and the choroid. The suprachoroidal space can also include the region between the sclera and the ciliary body. In this regard, the region of the suprachoroidal space between the sclera and the ciliary body may sometimes be referred to as the supraciliary space. The shunt SH need not be positioned between the choroid and the sclera. The shunt SH can be positioned at least partially between the ciliary body and the sclera or it can be at least partially positioned between the sclera and the choroid. In any event, the shunt SH provides a fluid pathway between the anterior chamber and the suprachoroidal space. The shunt SH as illustrated in FIG. 2 can be an elongate element having one or more internal lumens through which aqueous humor can flow from the anterior chamber AC into the suprachoroidal space.

These structures are, however, hidden from ordinary or direct view because of total internal reflection of light rays emanating from the angle structures. Viewing the angle for shunt implantation or evaluation, management and classification of normal and abnormal structures, generally requires the use of a viewing lens such as a gonioscope. Previous systems limited the continuous use of the gonioscope during steps of shunt implantation.

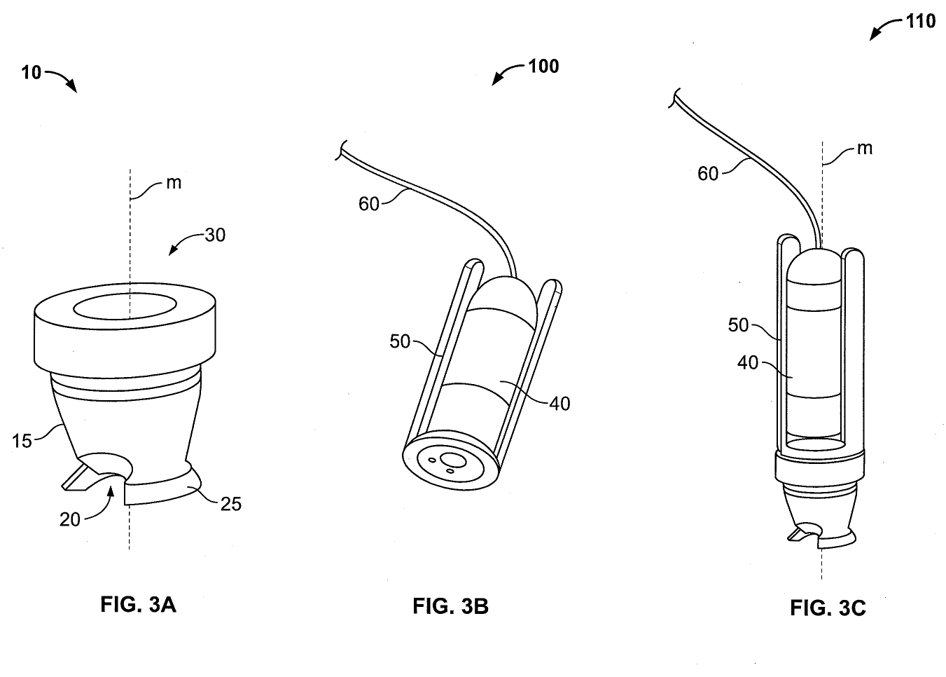

FIG. 3A shows an exemplary viewing lens 10. The viewing lens 10 includes a hollow tapered body 15 with one or more planar surfaces formed on the inner side that can have mirrored coatings. The mirrored surface(s) are arranged at select angles to assist in observation of the angle structures of the eye that are hidden from ordinary view. The viewing lens 10 also includes a corneal contact surface 20 that is typically a spherical, concave surface applied directly to the anterior surface of the cornea. The contact surface 20 has an optical axis M that can be aligned with the optical axis of the eye. The contact surface 20 can be smaller than the cornea so that the viewing lens 10 can be moved around on the cornea to view various parts of the eye. The viewing lens 10 can also have a flange 25 for stability. The flange 25 can have a double-radius such that the “step-down” makes contact with both the sclera and cornea.

The viewing lens 10 also has an anterior viewing surface 30 that extends in an anterior direction away from the contact surface 20. The viewing surface 30 has an optical axis that intersects the optical axis of the contact surface 20. The physician may view structures of the eye by looking into the viewing surface 30 in a direction generally parallel to the optical axis of the viewing surface 30. Typically, a surgical microscope (not shown) is used by the physician to peer through the viewing surface 30.

FIG. 3B shows a camera assembly 100 that can attach to the viewing lens 10 of FIG. 3A to provide an imaging system 110 shown in FIG. 3C. The assembly 100 includes a camera 40, a holder 50 and a data cable 60. The camera 40 can be, for example, a handheld USB digital microscope (Dino-Lite digital microscope) or a silicon-based CCD (charge-coupled device) digital color video camera or the like. The holder 50 couples the objective of the camera 40 to the viewing surface 30 of the viewing lens 10 such that the camera 40 is aligned generally parallel to the optical axis M of the viewing lens 10. However, this imaging system 110 limits the physician's view of the eye to the images provided by the camera 40 alone. Direct viewing through the viewing surface 30 of the viewing lens 10 itself by the physician is blocked by the positioning of the camera 40 along axis M.

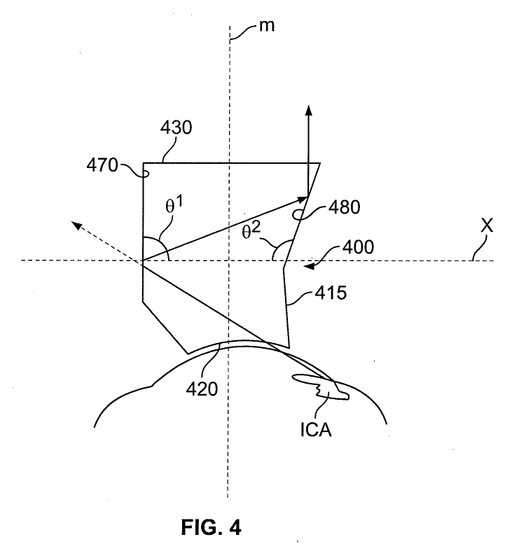

FIG. 4 shows an embodiment of a viewing lens that allows for simultaneous image capture and physician observation of structures inside a patient's eye, for example the iridocorneal angle (ICA) through the viewing surface of the viewing lens. The viewing lens 400 shown in FIG. 4 has a body 415 composed of an optically transparent material such as an optical grade glass or polymeric material. The corneal contact surface 420 has a concave surface centered on the optical axis M of the eye. The curvature of the contact surface 420 is similar to the convex curvature of the typical cornea of a patient. The anterior viewing surface 430 can be transverse to the optical axis M. The anterior viewing surface 430 can be planar or can carry a spherical curvature to increase the power of the optics. The exemplary viewing lens 400 can be a Mori gonioscope (Ocular Instruments, Bellevue, Wash. USA, see for example U.S. Pat. No. 6,976,758).

The viewing lens 400 can have two planar faces 470, 480 formed on the inner sides of the body 415. The faces 470, 480 extend from a location adjacent the proximal end of the body 415 and the periphery of the corneal contact surface 420 and extend radially outward in a distal direction relative to the optical axis M. The first planar surface 470 can have an angle θ¹ relative to a plane X orthogonal to the optical axis M of the eye (which in the case of the figure is also the optical axis of the viewing lens). This angle θ¹ may be varied depending upon the particular location in the eye that is desired to be observed. The second planar surface 480 can have an angle θ² relative to the plane X orthogonal to the optical axis M. Angles θ¹ and θ² can be, for example, between at least about 40 degrees and at least about 120 degrees. In an embodiment, angle θ² is at least about 110 degrees. In an embodiment, angle θ² is at least about 118 degrees. In an embodiment, angle θ¹ is at least about 80 degrees. In an embodiment, angle θ¹ is at least about 90 degrees.

The planar surfaces 470, 480 can be coated, such as with a mirrored coating. In an embodiment, the first planar surface 470 can be coated with a beam-splitting film material or coating. Beam-splitting films or coatings divide incident light into transmission and reflection components. The material transmits light of a first polarization and reflects a portion of light of a second polarization. This “half-mirror” allows for a portion of the light to be reflected towards the second planar surface 480 and another portion of the light to be transmitted through the side of the optically transparent body 415 of the viewing lens 400. The portion of light that is transmitted through the beam-splitting half-mirror 470 can be captured using an imaging device, as discussed in more detail below. The portion of light that is transmitted through the body 415 can vary, for example, the portion of light transmitted can be at least about 80%, 85%, 90%, or 95% of total light. In an embodiment, the portion of light transmitted through the body 415 is 92% of total light.

The second planar surface 480 is mirrored such that it reflects the image from the first planar surface 470 through the anterior viewing surface 430 towards the physician. The second mirrored surface 480 corrects the horizontal orientation of the image reflected from the first mirrored surface 470. This results in a more intuitive view of right-left movement for the purpose of instrument manipulation. Because the optical path is longer in the dual-mirror viewing lens compared to a single mirror lens or prism gonioscopes, the image appears to the physician to be more distant. The physician can use, for example, a surgical microscope in order to magnify the image. The surgical microscope worn by the physician can also provide added white light illumination as described in more detail below.

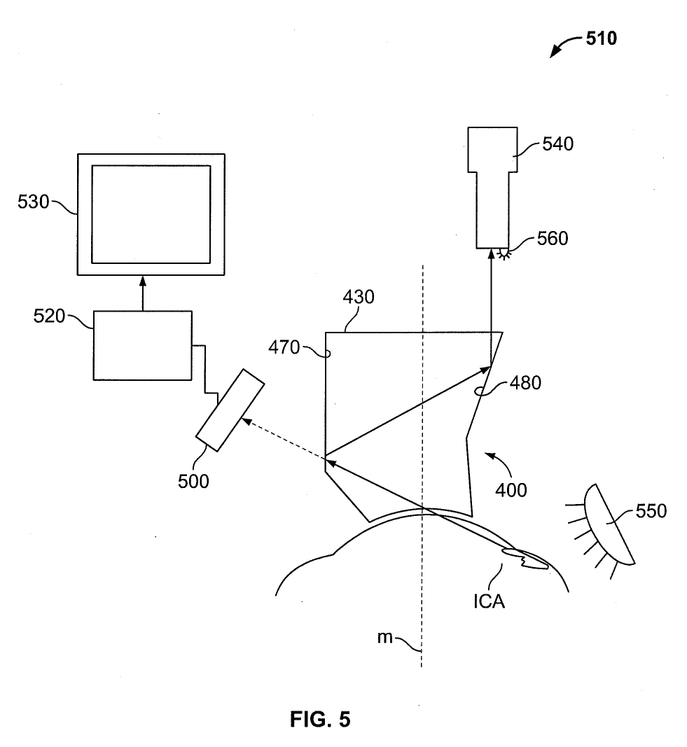

FIG. 5 shows a schematic representation of one embodiment of an imaging system 510. The imaging system 510 generally includes a viewing lens 400 and an image capture device 500 to collect light transmitted through the beam-splitting mirror 470 of the viewing lens 400. The system 510 can also include an evaluation module 520 and a data processing device 530 such as a PC computer for collecting, recording, and/or viewing image data. The image capture device 500 can be, for example, a handheld USB digital microscope (Dino-Lite digital microscope) or a silicon-based CCD (charge-coupled device) digital color video camera or the like. The image capture device 500 can be attached by, for example, a bracket, frame or holder system offset at an angle from the optical axis M of the viewing lens 400. The angle of the frame-mounted image capture device 500 is such that it does not block the physician's view through the anterior viewing surface 430 of the viewing lens 400 such as with a surgical microscope 540.

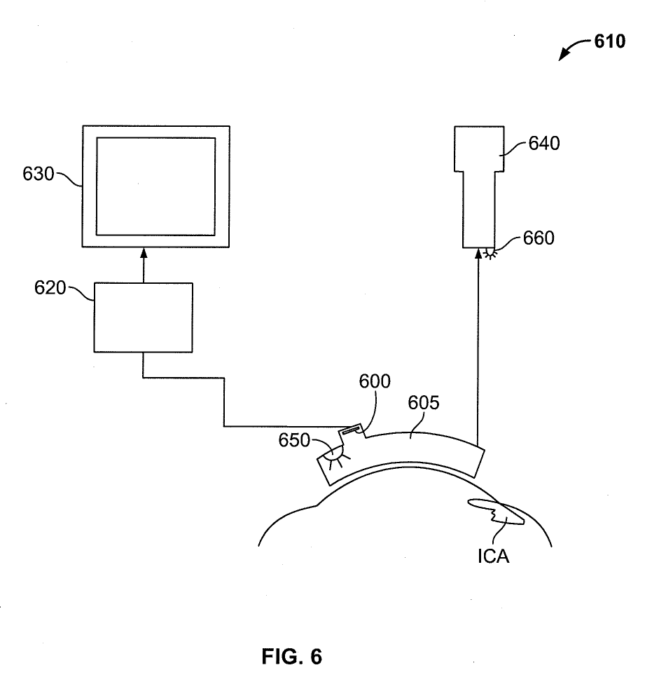

FIG. 6 shows a schematic representation of another embodiment of an imaging system 610. This imaging system 610 generally includes a viewing lens assembly 605 having an embedded image sensor 600. The imaging system 610 can also include an evaluation module 620 and a data processing device 630 as described above. The image sensor 600 collects light transmitted through a beam-splitting mirror of the viewing lens assembly 605. The image sensor 600 can be a low mass type of camera such as a CMOS (complementary metal oxide semiconductor) chip that is embedded directly into the viewing lens assembly 605, for example an OmniVision CMOS CameraChip™ image sensor. The portion of light transmitted through the beam-splitting mirrored surface of the viewing lens assembly 605 can be converted by the image sensor 600 into an electrical signal used by the evaluation module 620. The evaluation module 620 can be, for example an EFXB two board evaluation module that includes a flex module, EFA prototyping module and EAX USB 2.0 controller (OmniVision, Sunnyvale, Calif., USA).

In the embodiments shown in FIGS. 5 and 6 a source of infrared (IR) illumination is provided that can be picked up by the image capture device 500 or image sensor 600. In an embodiment, the IR illumination source can be an external IR illumination source 550 (as shown in FIG. 5). In another embodiment, the IR illumination source can be an embedded illumination source 650 (as shown in FIG. 6). IR illumination sources can include IR flood lamps, IR light-emitting diode (LED) and the like. The image capture device 500 or image sensor 600 can be sensitive to near infrared (IR) light in the 700-1200 nm (0.7-1.2 μm) range.

In addition to IR illumination for image capture with the image capture device 500 or image sensor 600, a white light illumination source can also be provided. For example, illumination sources 560, 660 such as incandescent or a white light LED can be provided for use with a surgical microscope 540, 640. Alternatively, fiberoptic light sources can be coupled to the periphery of the outer wall housing of the viewing lens. The source of white light illumination 560, 660 can be controlled independently of the source of IR illumination 550, 650 for the image capture device 500 or image sensor 600. This allows the physician to increase IR illumination in order to obtain the best IR image through the image capture device or sensor without affecting the physician's own view through the viewing lens using the visible light illumination source.

In an embodiment, images captured by the image capture device 500 or image sensor 600 can be processed directly by the data processing devices 530, 630. The image data can be visualized by the physician in real-time such as on a computer monitor for use during a procedure. The physician can simultaneously view image data through the viewing surface 430 of the viewing lens 400, 605 using, for example, a surgical microscope 540, 640 aided by illumination sources 560, 660 in the visible light spectrum. The data processing devices 530, 630 can be adapted to execute image analysis software and can include storage means for recording image data. The image data can include still images and/or video.

### EXEMPLARY METHODS OF DELIVERY AND IMPLANTATION

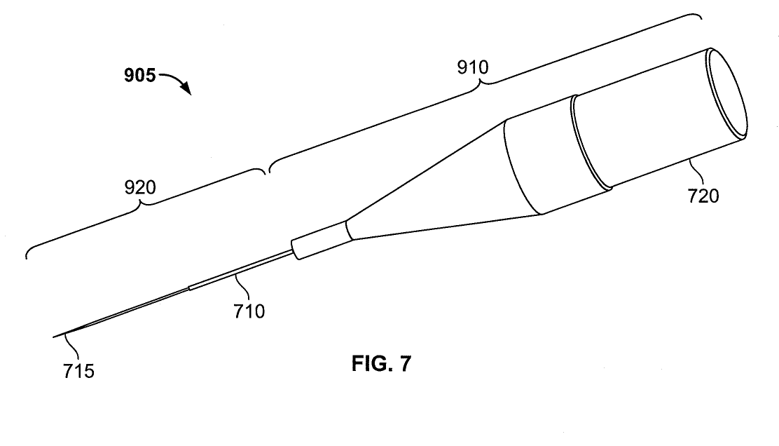

There are now described devices and methods for delivering and deploying an implant into the eye with the aid of imaging systems described herein. In an embodiment, a delivery system is used to deliver an implant into the eye such that the implant provides fluid communication between the anterior chamber and the suprachoroidal space. FIG. 7 shows an exemplary delivery system 905 that can be used to deliver the implant into the eye while viewing the target structures using an imaging system described above. It should be appreciated that the delivery system 905 is exemplary and that variations in the structure, shape and actuation of the delivery system 905 are possible. Each step of implantation can be continually visualized in real-time through either the viewing lens, for example with the aid of a surgical microscope and/or CCD camera or computer monitor.

The delivery system 905 generally includes a proximal handle component 910 that controls an implant placement mechanism and a distal delivery component 920 that removably couples to the implant for delivery of the implant into the eye. The delivery component 920 includes an elongate delivery wire 715 that is sized and shaped to be inserted longitudinally through the internal lumen of the implant. In one embodiment, the delivery wire 715 has a sharpened distal tip although it can also be blunt. The delivery wire 715 is sized to fit through the lumen in the implant such that the implant can be mounted on the delivery wire 715. The delivery wire 715 can have a cross-sectional shape that complements the cross-sectional shape of the internal lumen of the implant to facilitate mounting of the implant onto the delivery wire 715. The delivery wire 715 can be straight or it can be can be curved along all or a portion of its length in order to facilitate proper placement through the cornea. The delivery component 920 also includes a sheath 710 positioned axially over the delivery wire 715. The sheath 710 can aid in the release of the implant from the delivery component 920. The delivery system 905 can be actuated to achieve relative, sliding movement between the sheath 710 and the delivery wire 715.

With reference still to FIG. 7, the handle component 910 of the delivery system 905 can be actuated to control delivery of the implant. In this regard, the handle component 910 includes an actuator 720 that can be actuated to cause relative, sliding movement between the delivery wire 715 and the sheath 710. For example, the actuator 720 can be manipulated to cause the delivery wire 715 to withdraw proximally relative to the sheath 710. The actuator can vary in structure and mechanism and can include, for example, a button, switch, knob, slider, etc.

An exemplary method of delivering and implanting an implant into the eye using an imaging system is now described. In general, the implant is implanted using a delivery system by entering the eye through a corneal incision and penetrating the iris root or a region of the ciliary body or the iris root part of the ciliary body near its tissue border with the scleral spur to create a low-profile, minimally-invasive blunt dissection in the tissue plane between the choroid and the sclera. The implant is then positioned in the eye so that it provides fluid communication between the anterior chamber and the suprachoroidal space.

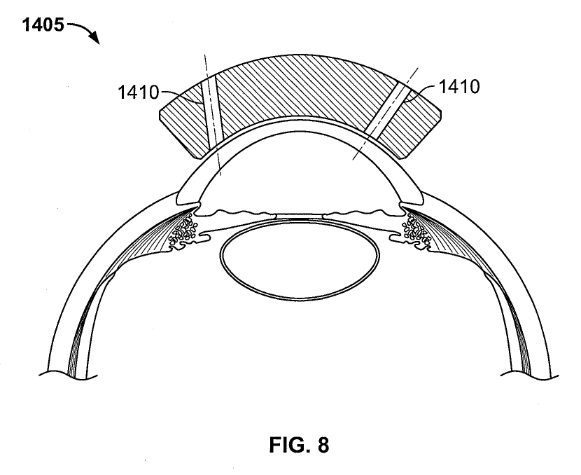

FIG. 8 shows a cross-sectional view of the eye. A viewing lens 1405, such as a goniolens, is positioned adjacent the cornea. The viewing lens 1405 can be part of an imaging system that enables real-time viewing of internal regions of the eye, such as the scleral spur and scleral junction, from a location in front of the eye during each step of implant delivery or other procedure. The viewing lens 1405 can optionally include one or more guide channels 1410 that are sized to receive the delivery portion 920 of the delivery system 905. It should be appreciated that the locations and orientations of the guide channels 1410 in FIG. 8 are merely exemplary and that the actual locations and orientations can vary depending on the angle and location where the implant 105 is to be delivered. The viewing lens 1405 can have a shape or cutout that permits the surgeon to use the viewing lens 1405 in a manner that does not cover or impede access to the corneal incision. Further, the viewing lens 1405 can act as a guide through which a delivery system 905 can be placed to predetermine the path of the device as it is inserted through the cornea.

An endoscope can also be used during delivery to aid in visualization. For example, a twenty-one to twenty-five gauge endoscope can be coupled to the implant during delivery such as by mounting the endoscope along the side of the implant or by mounting the endoscope coaxially within the implant. Ultrasonic guidance can be used as well using high resolution bio-microscopy, OCT and the like. Alternatively, a small endoscope can be inserted though another limbal incision in the eye to image the tissue during the procedure.

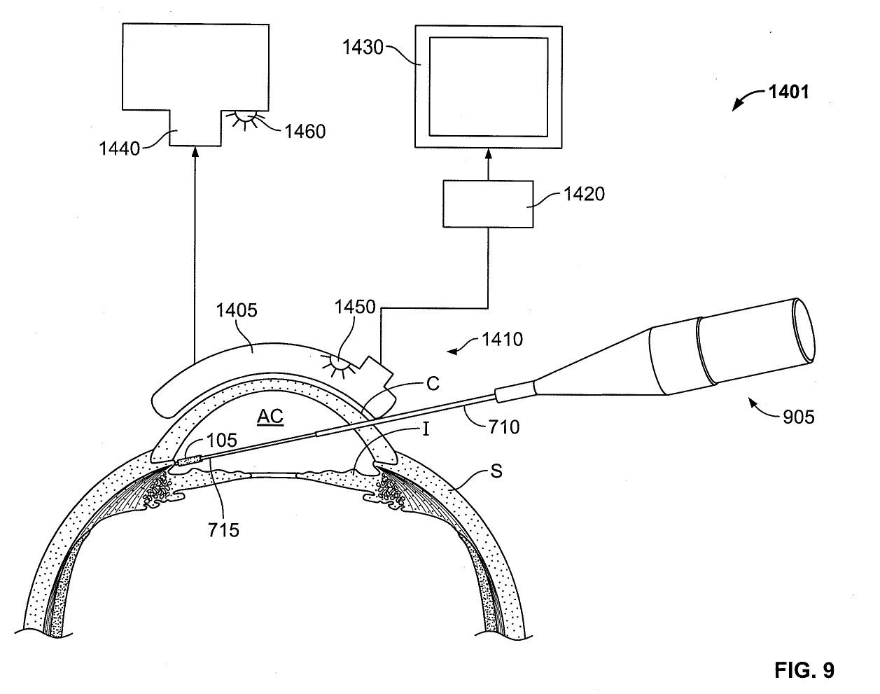

With respect to FIG. 9, one or more implants 105 can be mounted on the delivery system 905 for delivery into the eye. The eye can be viewed using imaging system 1401, in order to ascertain the location where the implant 105 is to be delivered and the accuracy with which the delivery is being performed. The imaging system 1401 is configured such that instrument manipulation as viewed through the viewing lens 1405 or other viewing means appears to the user to move in the same horizontal orientation as the instrument is actually being moved in space. This results in a more intuitive view of right-left movement for the purpose of instrument manipulation. At least one goal is to accurately deliver the implant 105 in the eye so that it is positioned such that the internal lumen of the implant provides a fluid pathway between the anterior chamber and the suprachoroidal space and delivery does not require removal of any component of the digital imaging system during delivery of the implant 105.

Still with reference to FIG. 9, the delivery system 905 is positioned such that the distal tip of the delivery wire 715 or the implant 105 itself can penetrate through the cornea. In this regard, an incision is made through the eye, such as within the limbus of the cornea. In an embodiment, the incision is very close to the limbus, such as either at the level of the limbus or within 2 mm of the limbus in the clear cornea. The delivery wire 715 can be used to make the incision or a separate cutting device can be used. For example, a knife-tipped device or diamond knife can be used to initially enter the cornea. A second device with a spatula tip can then be advanced over the knife tip wherein the plane of the spatula is positioned to coincide with the dissection plane. Thus, the spatula-shaped tip can be inserted into the suprachoroidal space with minimal trauma to the eye tissue.

The incision has a size that is sufficient to permit passage of the implant therethrough. In this regard, the incision can be sized to permit passage of only the implant without any additional devices, or be sized to permit passage of the implant in addition to additional devices, such as the delivery device or an imaging device. In an embodiment, the incision is about 1 mm in size. In another embodiment, the incision is no greater than about 2.85 mm in size. In another embodiment, the incision is no greater than about 2.85 mm and is greater than about 1.5 mm. It has been observed that an incision of up to 2.85 mm is a self-sealing incision. For clarity of illustration, the drawing is not to scale and the viewing lens 1405 is shown in FIG. 9 without guide channels, although the applier can be guided through one or more guide channels in the viewing lens.

The delivery wire 715 can approach the suprachoroidal space from the same side of the anterior chamber as the deployment location such that the applier does not have to be advanced across the iris. Alternately, the applier can approach the location from across the anterior chamber such that the applier is advanced across the iris and/or the anterior chamber (such as shown in the figure). The delivery wire 715 can approach the eye and the suprachoroidal space along a variety of pathways.

After insertion through the incision, the delivery wire 715 is advanced through the cornea and the anterior chamber. The applier is advanced along a pathway that enables the implant to be delivered to a position such that the implant provides a flow passageway from the anterior chamber to the suprachoroidal space. In one embodiment, the applier travels along a pathway that is toward the scleral spur such that the applier passes near the scleral spur on the way to the suprachoroidal space. In a preferred embodiment, the applier has a blunt tip that does not pass through the scleral spur during delivery. Rather, the applier abuts the scleral spur and then moves downward to dissect the tissue boundary between the sclera and the ciliary body, the dissection entry point starting just below the scleral spur. In an embodiment, the delivery wire 715 penetrates the iris root or a region of the ciliary body or the iris root part of the ciliary body near its tissue border with the scleral spur. The combination of blunt tipped instrument and approach allows the procedure to be performed “blind” as the instrument tip follows the inner curve of the scleral wall to dissect the tissue and create a mini cyclo-dialysis channel to connect the anterior chamber to the suprachoroidal space. The delivery wire 715 can be pre-shaped, steerable, articulating, or shapeable in a manner that facilitates the applier approaching the suprachoroidal space along a proper angle or pathway.

FIG. 9 shows a schematic of an embodiment of an imaging system 1401. The imaging system 1401 is shown in the figure as including a viewing lens assembly 1405 having an embedded image sensor 1410, an evaluation module 1420, and a data processing device 1430. It should be appreciated that the evaluation module 1420 and data processing device 1430 are optional. A portion of light is transmitted through a beam-splitting mirrored surface of the viewing lens 1405 and is converted by the image sensor 1410 into an electrical signal used by the evaluation module 1420. An illumination source 1450, such as an embedded IR LED, is also shown. The illumination source 1450 can have controls such that the surgeon can increase or decrease the amount of illumination such as IR light being exposed to the treatment area.

During each step of implantation, images of the structures and devices within the eye can be captured by the embedded image sensor 1410 to be viewed and/or recorded by the data processing device 1430. Simultaneously, the images of the structures and devices within the eye can be viewed by a surgeon such as through a surgical microscope 1440. As described in more detail above, the surgeon can view the surgical field using a surgical microscope 1440 that can have its own source of illumination 1460, such as white light illumination. Therefore, increasing IR illumination for better sensitivity through the image sensor 1410 does not affect a surgeon's direct view using, for example a surgical microscope 1440 and white light illumination 1460.

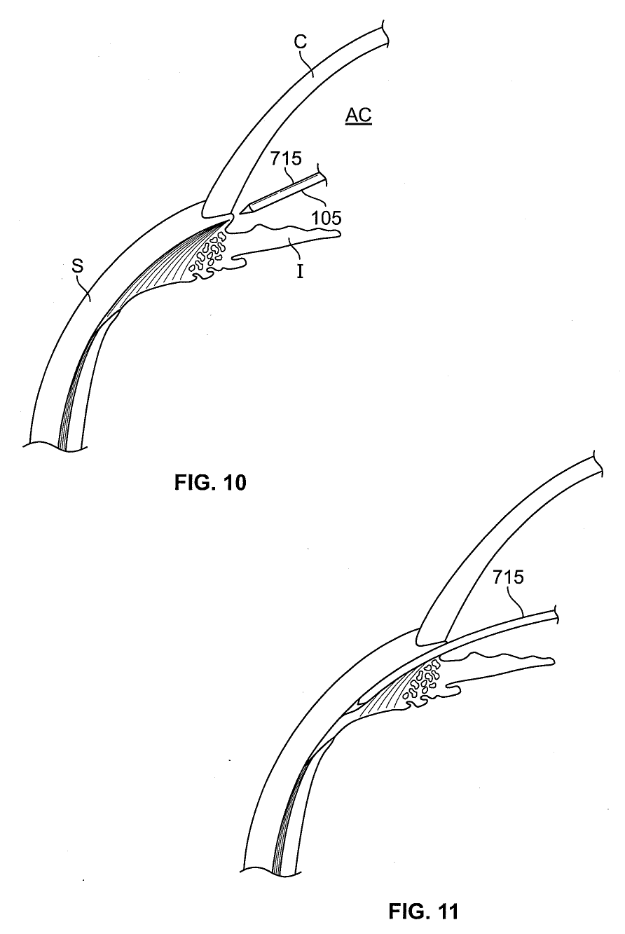

FIG. 10 shows an enlarged view of the anterior region of the eye. The implant 105 mounted on the delivery wire 715 can approach from the anterior chamber. They move along a pathway such that the dissection entry point of the distal tip of the delivery wire 715 can penetrate the iris root near its junction with the scleral spur or the iris root portion of the ciliary body. The scleral spur is an anatomic landmark on the wall of the angle of the eye. The scleral spur is above the level of the iris but below the level of the trabecular meshwork. In some eyes, the scleral spur can be masked by the lower band of the pigmented trabecular meshwork and be directly behind it. The surgeon can rotate or reposition the handle of the delivery device in order to obtain a proper approach trajectory for the distal tip of the applier, as described in further detail below. With the delivery wire 715 positioned for approach, the delivery wire 715 is then advanced further into the eye such that the distal tip of the applier and/or the implant penetrates the iris root near its junction with the scleral spur or the iris root portion of the ciliary body.

In an embodiment, the scleral spur can be penetrated during delivery. If penetration of the scleral spur does occur, penetration through the scleral spur can be accomplished in various manners. In one embodiment, a sharpened distal tip of the applier or the implant punctures, penetrates, dissects, pierces or otherwise passes through the scleral spur toward the suprachoroidal space. The crossing of the scleral spur or any other tissue can be aided such as by applying energy to the scleral spur or the tissue via the distal tip of the delivery wire 715. The means of applying energy can vary and can include mechanical energy, such as by creating a frictional force to generate heat at the scleral spur. Other types of energy can be used, such as RF laser, electrical, etc.

The delivery wire 715 is continuously advanced into the eye, via the trabecular meshwork and the ciliary body. The dissection plane of the delivery wire 715 follows the curve of the inner scleral wall such that the implant mounted on the delivery wire 715 after penetrating the iris root bluntly dissects the boundary between tissue layers of the scleral spur and the ciliary body such that a distal region of the implant extends into the suprachoroidal space. A proximal portion of the implant remains within the anterior chamber. In one embodiment, at least 1 mm to 2 mm of the implant (along the length) remains in the anterior chamber. FIG. 11 shows the distal tip of the delivery wire 715 positioned within the suprachoroidal space. For clarity of illustration, FIG. 11 does not show the implant mounted on the applier, although the implant is mounted on the applier during delivery. As the delivery wire 715 advances through tissue, the distal tip causes the sclera to peel away or otherwise separate from the ciliary body and the choroid to enter the suprachoroidal space. The delivery wire 715 and implant are then advanced into the suprachoroidal space such that at least the distal section of the implant is positioned in the suprachoroidal space and the proximal section remains in the anterior chamber. The implant is then released from the delivery wire 715. Each step of implantation can be visualized using the imaging systems described herein. Visualization using the imaging systems described herein can occur continuously during implantation or other procedures without the need for re-positioning or removing one or more components of the imaging systems (such as the viewing lens).

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

What is claimed:

1. A hand-held system for viewing the interior of a patient's eye, comprising:
a viewing lens having a corneal contact surface, an anterior viewing surface and an optically transparent body therebetween, the optically transparent body comprising a first internal planar surface and a second internal planar surface, the first and second internal planar surfaces each having a mirrored coating;
an illumination source; and
an imaging device.

2. The system of claim 1, further comprising a data processing device.

3. The system of claim 1, wherein the viewing lens comprises a goniolens.

4. The system of claim 1, wherein the mirrored coating of the first internal planar surface is configured to transmit at least a portion of light reflected from the interior of a patient's eye through the optically transparent body and reflect at least a portion of light towards the second internal planar surface.

5. The system of claim 4, wherein the mirrored coating of the first internal planar surface comprises a beam-splitting film.

6. The system of claim 4, wherein the second internal planar surface is configured to reflect in an anterior direction through the anterior viewing surface the portion of light reflected from the first internal planar surface such that the horizontal orientation of the reflected light from the first internal planar surface is reversed.

7. The system of claim 1, wherein the viewing lens has a central axis that aligns with the optical axis of the eye and wherein the first internal planar surface has an angle of at least about 90 degrees from a plane orthogonal to the central axis.

8. The system of claim 1, wherein the viewing lens has a central axis that aligns with the optical axis of the eye and wherein the second internal planar surface has an angle of at least about 110 degrees from a plane orthogonal to the central axis.

9. The system of claim 1, wherein the illumination source emits infrared light.

10. The system of claim 9, wherein the illumination source is embedded in the viewing lens.

11. The system of claim 10, wherein the illumination source is an LED.

12. The system of claim 9, wherein the illumination source is external to the system.

13. The system of claim 12, wherein the illumination source is a flood lamp.

14. The system of claim 1, further comprising a second illumination source.

15. The system of claim 14, wherein the second illumination source emits white light.

16. The system of claim 15, wherein the white light is incandescent, an LED or a fiberoptic light.

17. The system of claim 14, further comprising a first control mechanism configured to control the first illumination source and a second control mechanism configured to control the second illumination source, wherein the second illumination source control mechanism is independent of the first illumination source control mechanism.

18. The system of claim 4, wherein the imaging device is configured to capture the portion of light transmitted through the first internal planar surface.

19. The system of claim 18, wherein the imaging device is configured to capture video images or still images or both.

20. The system of claim 2, wherein the data processing device is configured to display an image from the imaging device in real-time.

21. The system of claim 2, wherein the data processing device is configured to record an image from the imaging device.

22. The system of claim 1, wherein the imaging device is selected from the group consisting of a hand-held digital microscope, a digital camera, a CCD video camera, a low mass camera, and a CMOS chip.

23. The system of claim 1, wherein the imaging device is embedded in the viewing lens.

24. The system of claim 1, wherein the viewing lens comprises a corneal contact surface having a double-radius flange.
